(19) United States
(12) Patent Application Publication
(10) Pub. No.: US 2022/0330901 A1
(43) Pub. Date: Oct. 20, 2022

(54) METHOD AND DETECTING DEVICE FOR DETERMINING TIREDNESS OF USER

(71) Applicant: National Chung-Hsing University, Taichung City (TW)

(72) Inventors: Kuan-Jiuh Lin, Taichung City (TW); Jun-Wei Su, Chiayi City (TW)

(73) Assignee: National Chung-Hsing University, Taichung City (TW)

(21) Appl. No.: 17/659,257

(22) Filed: Apr. 14, 2022

(30) Foreign Application Priority Data
Apr. 16, 2021 (TW) 110113689

(51) Int. Cl. A61B 5/00; A61B 5/024; A61B 5/22

(52) U.S. Cl. CPC A61B 5/7275; A61B 5/681; A61B 5/02438; A61B 5/7435; A61B 5/7475; A61B 5/746; A61B 5/222; A61B 2560/02

(57) ABSTRACT

This disclosure provides a method for determining tiredness of a user, which utilizes heart rate measurements, speed measurements and a plurality of predetermined parameters to determine whether the user is exercising and to determine how tired the user is if the user is determined to be exercising. A notification is outputted when it is determined that the user is tired.

### CROSS-REFERENCE TO RELATED APPLICATION

This application claims priority of Taiwanese Patent Application No. 110113689, filed on Apr. 16, 2021.

### FIELD

The disclosure relates to a method for determining tiredness of a user according to human physiological information.

### BACKGROUND

As smart wearable devices become more and more popular, smart watches and smart bracelets are now accessories that many people wear on a daily basis, and a lot of these people also use the physiological data recorded by the smart watches or bracelets as a reference for personal health management.

A conventional smart watch usually records heart rate as the basis for subsequent analysis of exercise time and calorie consumption. However, as the heart rate is being measured and recorded, the conventional smart watch is unable to provide feedback (e.g., excessive tiredness) in real time to the user, and thus the user cannot suitably adjust or stop the exercise in time. The user can only know his/her physical state when checking the conventional smart watch later.

A conventional assisting system for mobile vehicles, as disclosed in Taiwanese Utility Model Patent No. M585412, includes a driver condition detecting device and a warning device. The driver condition detecting device includes a heart rate detecting module, a storage module and a computing module. The heart rate detecting module is used for detecting heart rate of a driver or change in heart rate of the driver, and can be disposed on a wearable device to be worn by the driver.

Although the conventional assisting device can issue a notification when the heart rate of the user is outside of a predetermined range, it is prone to making inappropriate notifications when the user is exercising.

### SUMMARY

Therefore, an object of the disclosure is to provide a method for determining tiredness of a user that can alleviate at least one of the drawbacks of the prior art.

According to one embodiment of the disclosure, the method is implemented by a detecting device that stores a plurality of exercise modes respectively defined by a plurality of first ranges of heart-rate-related ratios, and each of the exercise modes has a variation threshold and a predetermined exercise time period. The method includes steps of:

measuring heart rate of the user at successive time instances so as to result in a plurality of heart rate measurements;

for each of the heart rate measurements, calculating a reference ratio as a ratio of a resulting value of the heart rate measurement minus a resting heart rate of the user to a resulting value of a maximum heart rate of the user minus the resting heart rate;

selecting one of the exercise modes based on the reference ratio calculated based on a last one of the heart rate measurements;

obtaining a plurality of speed measurements of the user that were successively measured in a past time period having a length equal to the predetermined exercise time period of said one of the exercise modes;

calculating an average of the speed measurements to serve as an average speed;

dividing a number of those of the speed measurements that are greater or smaller than the average speed at least by a variation ratio by a total number of the speed measurements to obtain an abnormal speed ratio;

determining whether the average speed is greater than a predetermined speed and whether the abnormal speed ratio is smaller than a predetermined ratio;

when the determination on whether the average speed is greater than the predetermined speed and the determination on whether the abnormal speed ratio is smaller than the predetermined ratio are both affirmative, determining whether the reference ratio that was calculated based on the last one of the heart rate measurements is greater than the reference ratio that was calculated based on a previous one of the heart rate measurements at least by the variation threshold of said one of the exercise modes, wherein the previous one of the heart rate measurements was measured earlier than the last one of the heart rate measurements by the predetermined exercise time period of said one of the exercise modes; and

outputting a notification indicating that the user is tired when it is determined that the reference ratio that was calculated based on the last one of the heart rate measurements is greater than the reference ratio that was calculated based on the previous one of the heart rate measurements at least by the variation threshold of said one of the exercise modes.

Another object of the disclosure is to provide a detecting device for monitoring heart rate of a user.

According to one embodiment of the disclosure, the monitoring device includes a heart rate measuring unit, a positioning unit, a display unit and a processing unit.

The heart rate measuring unit is configured to measure heart rate of the user at successive time instances so as to result in a plurality of heart rate measurements.

The positioning unit is configured to detect where the user is located at successive time instances so as to obtain a plurality of positions, and is further configured to calculate a plurality of speed measurements of the user based on the positions.

The processing unit is electrically connected to the heart rate measuring unit, the positioning unit and the display unit, and stores a plurality of exercise modes respectively defined by a plurality of ranges of heart-rate-related ratios. Each of the exercise modes has a variation threshold and a predetermined exercise time period. The processing unit is configured to implement a tiredness-determining procedure including steps of:

obtaining the heart rate measurements of the user from the heart rate measuring unit;

for each of the heart rate measurements, calculating a reference ratio as a ratio of a resulting value of the heart rate measurement minus a resting heart rate of the user to a resulting value of a maximum heart rate of the user minus the resting heart rate;

selecting one of the exercise modes based on the reference ratio that was calculated based on a last one of the heart rate measurements;

obtaining, from the positioning unit, a plurality of speed measurements that were successively measured by the positioning unit in a past time period having a length equal to the predetermined exercise time period of said one of the exercise modes;

calculating an average of the speed measurements to serve as an average speed;

dividing a number of those of the speed measurements that are greater or smaller than the average speed at least by a variation ratio by a total number of the speed measurements to obtain an abnormal speed ratio;

determining whether the average speed is greater than a predetermined speed and whether the abnormal speed ratio is smaller than a predetermined ratio;

when the determination on whether the average speed is greater than the predetermined speed and the determination on whether the abnormal speed ratio is smaller than the predetermined ratio are both affirmative, determining whether the reference ratio that was calculated based on the last one of the heart rate measurements is greater than the reference ratio that was calculated based on a previous one of the heart rate measurements at least by the variation threshold of said one of the exercise modes, wherein the previous one of the heart rate measurements was measured earlier than the last one of the heart rate measurements by the predetermined exercise time period of said one of the exercise modes; and

controlling the display unit to output a notification indicating that the user is tired when it is determined that the reference ratio that was calculated based on the last one of the heart rate measurements is greater than the reference ratio that was calculated based on the previous one of the heart rate measurements at least by the variation threshold of said one of the exercise modes.

### BRIEF DESCRIPTION OF THE DRAWINGS

Other features and advantages of the disclosure will become apparent in the following detailed description of the embodiments with reference to the accompanying drawings, of which:

FIG. 1 is a flow chart illustrating steps of a method for determining tiredness of a user according to an embodiment of the disclosure;

FIG. 2 is a block diagram of a detecting device for determining tiredness of the user according to an embodiment of the disclosure;

FIG. 3 is a chart of heart rate measurements, for illustrating an example of determining exercise modes according to an embodiment of the disclosure; and

FIG. 4 is a chart of heart rate measurements, for illustrating an example of determining tiredness levels when the user is resting according to an embodiment of the disclosure.

### DETAILED DESCRIPTION

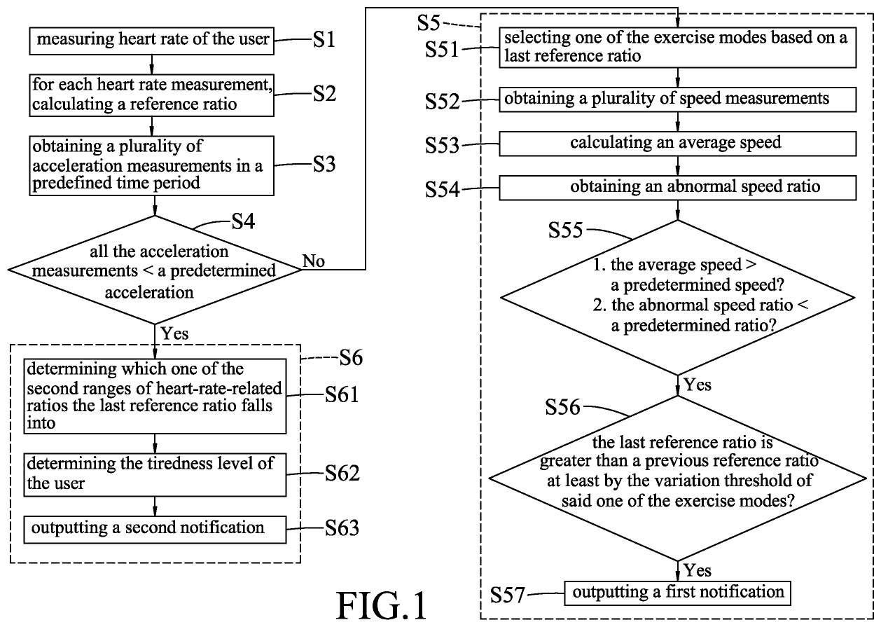

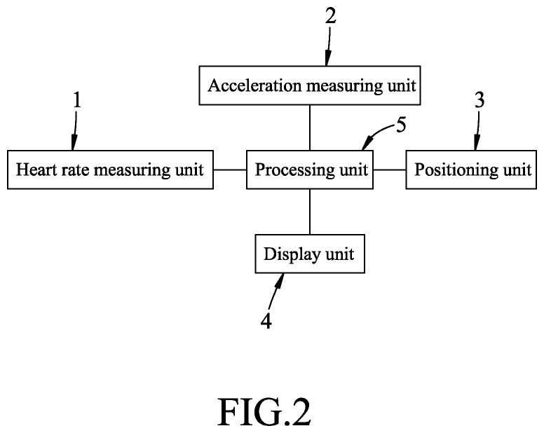

FIG. 1 is a flow chart illustrating steps of a method for determining tiredness of a user according to one embodiment of the disclosure. The method is implemented by a detecting device shown in FIG. 2.

Referring to FIG. 2, the detecting device includes a heart rate measuring unit 1, an acceleration measuring unit 2, a positioning unit 3, a display unit 4 and a processing unit 5. The detecting device may be, but not limited to, a wearable device worn by the user.

The heart rate measuring unit 1 is configured to measure heart rate of the user at successive time instances so as to result in a plurality of heart rate measurements. In this embodiment, the heart rate measuring unit 1 is an optical heart rate sensor.

The acceleration measuring unit 2 is configured to successively measure a plurality of acceleration measurements of the user. The acceleration measuring unit 2 may be, but not limited to, an accelerometer.

The positioning unit 3 is configured to detect where the user is located at successive time instances so as to obtain a plurality of positions, and is further configured to calculate a plurality of speed measurements of the user based on the positions. For example, the positioning unit 3 includes a global positioning system (GPS) sensor for obtaining the position of the user, and a computing device (e.g., a processor, a mobile processor, a microprocessor, a microcontroller, etc.) for calculating the speed measurements.

The display unit 4 is configured to output a notification. In this embodiment, the display unit 4 is, but not limited to, a display screen.

The processing unit 5 is electrically connected to the heart rate measuring unit 1, the acceleration measuring unit 2, the positioning unit 3 and the display unit 4. The processing unit 5 stores a plurality of exercise modes and a plurality of tiredness levels. The exercise modes are respectively defined by a plurality of first ranges of heart-rate-related ratios. The tiredness levels are respectively defined by a plurality of second ranges of heart-rate-related ratios. Each of the exercise modes has a variation threshold and a predetermined exercise time period.

In this embodiment, the processing unit 5 stores five exercise modes, and details thereof are shown in Table 1 below.

TABLE 1

| | | Predetermined | |
|---|---|---|---|
| Exercise | | Exercise Time | Variation |
| Mode | Definition | Period | Threshold |
| Exercise | reference | 120 minutes | 10% |
| Mode 1 | ratio ≤ 30% | | |
| Exercise | 30% < | 90 minutes | 10% |
| Mode 2 | reference | | |
| | ratio ≤ 50% | | |
| Exercise | 50% < | 30 minutes | 10% |
| Mode 3 | reference | | |
| | ratio ≤ 70% | | |
| Exercise | 70% < | 10 minutes | 10% |
| Mode 4 | reference | | |
| | ratio ≤ 90% | | |
| Exercise | 90% < | 5 minutes | 10% |
| Mode 5 | reference | | |
| | ratio | | |

In this embodiment, the processing unit 5 stores five tiredness levels, and details thereof are shown in Table 2 below.

TABLE 2

| Tiredness Level | Definition |
|---|---|
| not tired | reference ratio ≤ 2% |
| slightly tired | 2% < reference ratio ≤ 5% |
| tired | 5% < reference ratio ≤ 8% |
| very tired | 8% < reference ratio ≤ 11% |
| extremely tired | 11% < reference ratio |

The processing unit 5 is configured to implement a tiredness-determining procedure for determining tiredness of the user based on heart rate measurements, the acceleration measurements and the speed measurements of the user.

The processing unit 5 may be embodied using a central processing unit (CPU), a microprocessor, a microcontroller, a single core processor, a multi-core processor, a dual-core mobile processor, a microprocessor, a microcontroller, a digital signal processor (DSP), a field-programmable gate array (FPGA), an application specific integrated circuit (ASIC), a radio-frequency integrated circuit (RFIC), a system on chip (SoC), etc.

The method for determining tiredness of the user (i.e., the tiredness-determining procedure implemented by the processing unit 5) according to one embodiment includes the following steps.

In step S1, the processing unit 5 obtains a plurality of heart rate measurements of the user that were successively measured by the heart rate measuring unit 1.

In step S2, for each of the heart rate measurements, the processing unit 5 calculates a ratio of a resulting value of the heart rate measurement minus a resting heart rate of the user to a resulting value of a maximum heart rate of the user minus the resting heart rate to serve as a reference ratio.

The maximum heart rate is an upper limit of what a person's cardiovascular system can typically handle during exercise (i.e., the fastest rate that the heart is capable of beating), and generally decreases with age. The maximum heart rate may be estimated by subtracting age from 220. The resting heart rate is a normal heart rate when a person is at rest. The resting heart rates of children over 10 years old and adults including seniors range from 60 to 100 beats per minute. The resting heart rates of well-trained adult athletes range from 40 to 60 beats per minute. In this embodiment, the maximum heart rate and the resting heart rate may be input by the user. In some embodiments, the processing unit 5 may pre-store a plurality of reference maximum heart rates and a plurality of reference resting heart rates that correspond respectively to the reference maximum heart rates, and each pair of one of the reference maximum heart rates and the corresponding one of the reference resting heart rates corresponds respectively to different ages; the processing unit 5 selects one of the reference maximum heart rates and the corresponding one of the reference resting heart rates according to an age inputted by the user and uses the reference maximum heart rate and reference resting heart rate thus selected as the maximum heart rate and the resting heart rate of the user. In some embodiments, the resting heart rate may be obtained by taking an average of heart rate measurements measured by the heart rate measuring unit 1 when the acceleration of the user continues to be smaller than a predetermined value (e.g., 20 cm/s²) for a predetermined time period (e.g., 20 minutes).

In step S3, the processing unit 5 obtains, from the acceleration measuring unit 2, a plurality of acceleration measurements of the user measured in a predefined time period. In this embodiment, the predefined time period is 20 minutes.

In step S4, the processing unit 5 determines whether all the acceleration measurements in the predefined time period are smaller than a predetermined acceleration threshold. When it is determined that all the acceleration measurements are smaller than the predetermined acceleration threshold, the flow goes to step S6; otherwise, the flow goes to step S5. In this embodiment, the predetermined acceleration threshold is 20 cm/s².

It should be noted that step S4 is used to determine whether the user is exercising or resting. The user is determined to be resting when it is determined that all the acceleration measurements are smaller than the predetermined acceleration threshold.

When it is determined that not all the acceleration measurements in the predefined time period are smaller than the predetermined acceleration threshold, in step S5, the processing unit 5 determines tiredness of the user during exercise. Specifically, step S5 includes sub-steps S51 to S57.

In sub-step S51, the processing unit 5 selects one of the exercise modes based on the reference ratio that was calculated in step S2 based on a last one of the heart rate measurements (hereinafter referred to as “the last reference ratio”). Specifically, the processing unit 5 first determines which one of the first ranges of heart-rate-related ratios the last reference ratio falls into, and then selects one of the exercise modes that corresponds to one of the first ranges of heart-rate-related ratios, into which the last reference ratio falls.

In sub-step S52, the processing unit 5 obtains a plurality of speed measurements of the user that were successively measured by the positioning unit 3 in a past time period that has a length equal to the predetermined exercise time period of said one of the exercise modes.

In sub-step S53, the processing unit 5 calculates an average of the speed measurements obtained in sub-step S52 to serve as an average speed.

In sub-step S54, the processing unit 5 divides a number of those of the speed measurements that are greater or smaller than the average speed at least by a variation ratio by a total number of the speed measurements, so as to obtain an abnormal speed ratio. In this embodiment, the variation ratio is, but not limited to, 20%.

In sub-step S55, the processing unit 5 determines whether the average speed is greater than a predetermined speed and whether the abnormal speed ratio is smaller than a predetermined ratio. When the determination on whether the average speed is greater than the predetermined speed and the determination on whether the abnormal speed ratio is smaller than the predetermined ratio are both affirmative, the flow goes to sub-step S56; otherwise, the method is terminated. In this embodiment, the predetermined speed is 10 km/h, and the predetermined ratio is 1/6, but not limited thereto.

It should be noted that, in sub-step S55, determining whether the average speed is greater than the predetermined speed is to determine whether the user is moving places (e.g., doing an exercise such as jogging or cycling), and determining whether the abnormal speed ratio is smaller than the predetermined ratio is to avoid determining tiredness of the user when the speed measurements reveal an excessive change in speed. That is to say, sub-step S55 can avoid determining tiredness of the user when the user is in the midst of speeding up or slowing down, so as to reduce probability of making an incorrect determination on tiredness of the user.

When the average speed is greater than the predetermined speed and the abnormal speed ratio is smaller than the predetermined ratio, in sub-step S56, the processing unit 5 determines whether the last reference ratio is greater than a previous one of the reference ratios (hereinafter referred to as “previous reference ratio”) at least by the variation threshold of said one of the exercise modes, wherein the previous reference ratio is calculated based on one of the heart rate measurements that was measured earlier than said last one of the heart rate measurements by the predetermined exercise time period of said one of the exercise modes. When it is determined that the last reference ratio is greater than the previous reference ratio at least by the variation threshold of said one of the exercise modes, the flow goes to sub-step S57; otherwise, the method is terminated.

In sub-step S57, the processing unit 5 controls the display unit 4 to output a first notification indicating that the user is tired.

When it is determined in step S4 that all the acceleration measurements are smaller than the predetermined acceleration threshold, in step S6, the processing unit 5 determines tiredness of the user when resting. Specifically, step S6 includes sub-steps of S61 to S63.

In sub-step S61, the processing unit 5 determines which one of the second ranges of heart-rate-related ratios the last reference ratio falls into.

In sub-step S62, the processing unit 5 determines that the user is at one of the tiredness levels that corresponds to one of the second ranges of heart-rate-related ratios, into which the last reference ratio falls.

In sub-step S63, the processing unit 5 controls the display unit 4 to output a second notification that corresponds to said one of the tiredness levels.

The method of the disclosure may be repeated to continuously determine tiredness of the user. It should be noted that steps/sub-steps of the method are not necessarily implemented in the order given above, and some of the steps/sub-steps may be implemented simultaneously.

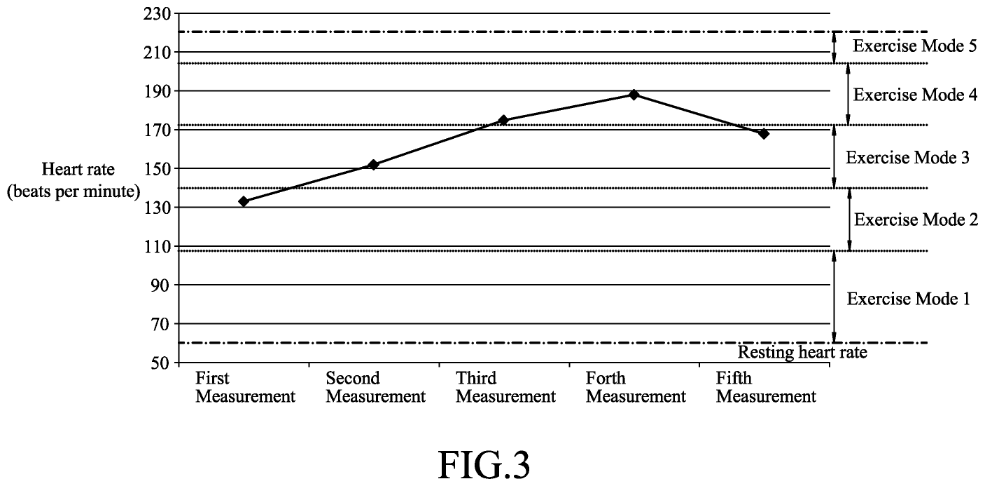

Referring to FIGS. 1 and 3 and Table 3 below, an exemplary flow for determining one of the exercise modes is described in the following. When it is determined in step S4 that at least one of the acceleration measurements of the user in the predefined time period (20 minutes) is greater than the predetermined acceleration threshold (20 cm/s²), and it is determined in sub-step S55 that the average speed is greater than the predetermined speed (10 km/h) and the abnormal speed ratio is smaller than the predetermined ratio (1/6), the flow goes to sub-step S56 to determine whether the last reference ratio is greater than the previous reference ratio. For example, five heart rate measurements of 133, 152, 175, 188 and 168 are obtained in step S1 in sequence, and the maximum heart rate is 220 and the resting heart rate is 60. Accordingly, the reference ratios for the five heart rate measurements are 46%, 58%, 72%, 80% and 68% (step S2), respectively, and the exercise modes corresponding to the five heart rate measurements are Exercise Mode 2, Exercise Mode 3, Exercise Mode 4, Exercise Mode 4 and Exercise Mode 5 (see Table 1), respectively. It should be noted that there may be one or more heart rate measurements that were measured between any two of the first to fifth heart rate measurements and that are not shown in this example.

TABLE 3

| | First | Second | Third | Forth | Fifth |
|---|---|---|---|---|---|
| Heart | 133 | 152 | 175 | 188 | 168 |
| Rate | | | | | |
| Ref. | 46% | 58% | 72% | 80% | 68% |
| Ratio | | | | | |
| Exercise | Mode 2 | Mode 3 | Mode 4 | Mode 4 | Mode 3 |
| Mode | | | | | |

Taking the second heart rate measurement as an example, the exercise mode that corresponds to the second heart rate measurement is Exercise Mode (with reference to Table 1), so sub-step S56 is to determine whether the reference ratio of the second heart rate measurement (58%) is greater than the previous reference ratio, which corresponds to a heart rate measurement that was measured 30 minutes (i.e., the predetermined exercise time period of Exercise Mode 3) earlier, at least by 10%. It is assumed that the first heart rate measurement of 133 was measured earlier than the second heart rate measurement of 152 by the predetermined exercise time period (i.e., 30 minutes), so the previous reference ratio is 46%. Compared with the previous reference ratio (46%), the reference ratio of the second heart rate measurement (58%) has an increase of 12%, which is more than 10% (the variation threshold of Exercise mode 3). Accordingly, the display unit 4 of the detecting device outputs the first notification indicating that the user is tired (sub-step S57).

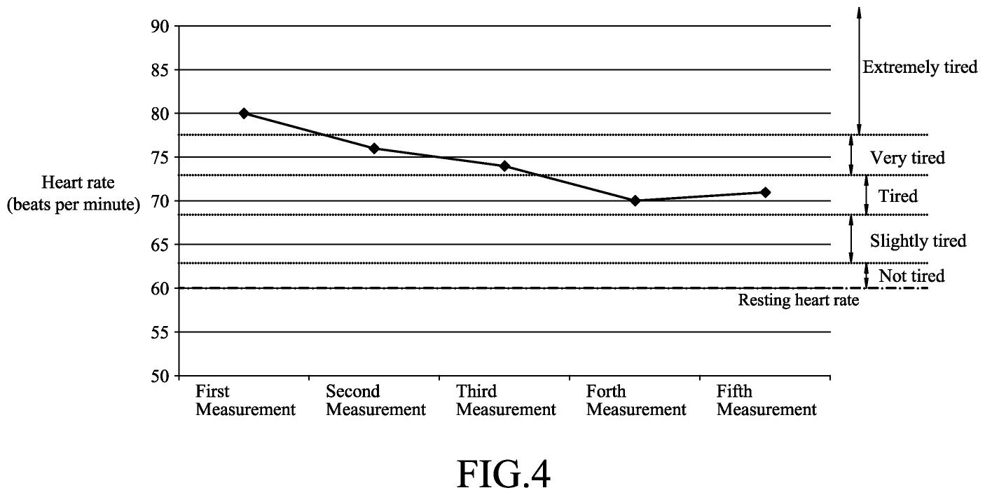

Referring to FIGS. 1 and 4 and Table 4, an exemplary flow for determining one of the tiredness levels of the user is described in the following. When all the acceleration measurements of the user in the predefined time period (20 minutes) are smaller than the predetermined acceleration threshold (20 cm/s²), the flow goes to step S6 to determine tiredness of the user when resting. For example, five heart rate measurements of 80, 76, 74, 70 and 71 are obtained in step S1 in sequence, and the maximum heart rate is 220 and the resting heart rate is 60. Accordingly, the reference ratios for the five heart rate measurements are 13%, 10%, 9%, 6%, and 7%, respectively (step S2), and the tiredness levels corresponding to the five heart rate measurements are “extremely tired”, “very tired”, “very tired”, “tired” and “tired”, respectively. Specifically, for the second heart rate measurement of 76, the reference ratio of 10% falls into the second range of heart-rate-related ratio “8%<reference ratio≤11%” (see Table 2), and thus the tiredness level of the second heart rate measurement is determined as “very tired”. It should be noted that there may be one or more heart rate measurements that were measured between any two of the first to fifth heart rate measurements and that are not shown in this example.

TABLE 4

| | First | Second | Third | Forth | Fifth |
|---|---|---|---|---|---|
| Heart | 80 | 76 | 74 | 70 | 71 |
| Rate | | | | | |
| Reference | 13% | 10% | 9% | 6% | 7% |
| Ratio | | | | | |
| Tiredness | extremely | very | very | tired | tired |
| Level | tired | tired | tired | | |

It should be noted that, unless otherwise specified, the use of the ordinal adjectives “first,” “second,” and “third,” etc., to describe a common object, merely indicates that different instances of like objects are being referred to and does not intend to imply that the objects so described must be in a given sequence, either temporally, spatially, in ranking or in any other manner.

According to the above description, the embodiment of the disclosure provides a method and a detecting device for determining tiredness of a user. The advantages of the method and the detecting device are as follows. An exercise mode is first selected based on a reference ratio of a heart rate measurement, and thresholds for the subsequent determinations are dynamically determined according to the exercise mode, so that the likelihood of making incorrect determinations is reduced as compared to the case if uniform thresholds were used. Further, tiredness determination is made only under the condition that an average speed is greater than a predetermined speed and an abnormal speed ratio is smaller than a predetermined ratio, so as to avoid incorrectly determining tiredness of the user when the user is speeding up or slowing down. Moreover, the method first determines whether the user is exercising or resting based on acceleration measurements, and then adopts difference logical flows in determining whether the user is tired respectively during exercise and when resting, such that tiredness of the user thus determined is more in line with actual situation.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method of determining tiredness of a user, to be implemented by a detecting device that stores a plurality of exercise modes respectively defined by a plurality of first ranges of heart-rate-related ratios, each of the exercise modes having a variation threshold and a predetermined exercise time period, the method comprising steps of:
A) measuring heart rate of the user at successive time instances so as to result in a plurality of heart rate measurements;
B) for each of the heart rate measurements, calculating a ratio of a resulting value of the heart rate measurement minus a resting heart rate of the user to a resulting value of a maximum heart rate of the user minus the resting heart rate to serve as a reference ratio;
C) selecting one of the exercise modes based on the reference ratio that was calculated based on a last one of the heart rate measurements;
D) obtaining a plurality of speed measurements of the user that were successively measured in a past time period having a length equal to the predetermined exercise time period of said one of the exercise modes;
E) calculating an average of the speed measurements to serve as an average speed;
F) dividing a number of those of the speed measurements that are greater or smaller than the average speed at least by a variation ratio by a total number of the speed measurements to obtain an abnormal speed ratio;
G) determining whether the average speed is greater than a predetermined speed and whether the abnormal speed ratio is smaller than a predetermined ratio;
H) when the determination on whether the average speed is greater than the predetermined speed and the determination on whether the abnormal speed ratio is smaller than the predetermined ratio are both affirmative, determining whether the reference ratio that was calculated based on the last one of the heart rate measurements is greater than the reference ratio that was calculated based on a previous one of the heart rate measurements at least by the variation threshold of said one of the exercise modes, wherein the previous one of the heart rate measurements was measured earlier than the last one of the heart rate measurements by the predetermined exercise time period of said one of the exercise modes; and
I) outputting a first notification indicating that the user is tired when the determination made in step H) is affirmative.

2. The method of claim 1, further comprising, before step C), steps of:
J) obtaining a plurality of acceleration measurements of the user in a predefined time period;
K) determining whether all the acceleration measurements in the predefined time period are smaller than a predetermined acceleration threshold;
L) when it is determined that all the acceleration measurements in the predefined time period are smaller than the predetermined acceleration threshold, determining tiredness of the user and outputting a second notification related to the tiredness of the user based on the reference ratio that was calculated based on the last one of the heart rate measurements; and
M) when it is determined that not all the acceleration measurements in the predefined time period are smaller than the predetermined acceleration threshold, executing steps C) to I).

3. The method of claim 2, the detecting device further storing a plurality of tiredness levels respectively defined by a plurality of second ranges of heart-rate-related ratios, wherein step L) includes:
determining which one of the second ranges of heart-rate-related ratios the reference ratio that was calculated based on the last one of the heart rate measurements falls into; and
determining that the user is at one of the tiredness levels that corresponds to one of the second ranges of heart-rate-related ratios, into which the reference ratio that was calculated based on the last one of the heart rate measurements falls.

4. The method of claim 2, wherein the predefined time period is 20 minutes.

5. The method of claim 2, wherein the predetermined acceleration threshold is 20 cm/s².

6. The method of claim 1, wherein the variation ratio is 20%.

7. The method of claim 1, wherein the predetermined speed is 10 km/h, and the predetermined ratio is 1/6.

8. The method of claim 1, wherein the variation threshold of each of the exercise modes is 10%.

9. A detecting device for determining tiredness of a user, comprising:
a heart rate measuring unit configured to measure heart rate of the user at successive time instances so as to result in a plurality of heart rate measurements;
a positioning unit configured to detect where the user is located at successive time instances so as to obtain a plurality of positions, and further configured to calculate a plurality of speed measurements of the user based on the positions;
a display unit; and
a processing unit electrically connected to said heart rate measuring unit, said positioning unit, and said display unit, and storing a plurality of exercise modes respectively defined by a plurality of first ranges of heart-rate-related ratios, each of the exercise modes having a variation threshold and a predetermined exercise time period, said processing unit being configured to implement a tiredness-determining procedure that includes steps of
a) obtaining the heart rate measurements of the user from said heart rate measuring unit,
b) for each of the heart rate measurements, calculating a ratio of a resulting value of the heart rate measurement minus a resting heart rate of the user to a resulting value of a maximum heart rate of the user minus the resting heart rate to serve as a reference ratio,
c) selecting one of the exercise modes based on the reference ratio that was calculated based on a last one of the heart rate measurements,
d) obtaining, from said positioning unit, a plurality of speed measurements that were successively measured by said positioning unit in a past time period having a length equal to the predetermined exercise time period of said one of the exercise modes,
e) calculating an average of the speed measurements to serve as an average speed,
f) dividing a number of those of the speed measurements that are greater or smaller than the average speed at least by a variation ratio by a total number of the speed measurements to obtain an abnormal speed ratio,
g) determining whether the average speed is greater than a predetermined speed and whether the abnormal speed ratio is smaller than a predetermined ratio,
h) when the determination on whether the average speed is greater than the predetermined speed and the determination on whether the abnormal speed ratio is smaller than the predetermined ratio are both affirmative, determining whether the reference ratio that was calculated based on the last one of heart rate measurements is greater than the reference ratio that was calculated based on a previous one of heart rate measurements at least by the variation threshold of said one of the exercise modes, wherein the previous one of the heart rate measurements was measured earlier than the last one of the heart rate measurements, by the predetermined exercise time period of said one of the exercise modes, and
i) controlling said display unit to output a first notification indicating that the user is tired when the determination made in step h) is affirmative.

10. The detecting device of claim 9, further comprising an acceleration measuring unit electrically connected to said processing unit and configured to successively measure acceleration measurements of the user, wherein the tiredness-determining procedure implemented by said processing unit further includes steps, before step c), of:
obtaining, from said acceleration measuring unit, a plurality of acceleration measurements of the user measured in a predefined time period;
determining whether all the acceleration measurements in the predefined time period are smaller than a predetermined acceleration threshold;
when it is determined that all the acceleration measurements in the predefined time period are smaller than the predetermined acceleration threshold, determining tiredness of the user and controlling said display unit to output a second notification based on the reference ratio that was calculated based on the last one of the heart rate measurements; and
when it is determined that not all the acceleration measurements in the predefined time period are smaller than the predetermined acceleration threshold, executing steps c) to i) of the tiredness-determining procedure.

11. The detecting device of claim 10, wherein said processing unit further stores a plurality of tiredness levels respectively defined by a plurality of second ranges of heart-rate-related ratios, and is configured to determine tiredness of the user by:
determining which one of the second ranges of heart-rate-related ratios the last one of the reference ratios falls into; and
determining that the user is at one of the tiredness levels that corresponds to one of the second ranges of heart-rate-related ratios, into which the reference ratio that was calculated based on the last one of the heart rate measurements falls, and outputting the second notification that corresponds to said one of the tiredness levels.

12. The detecting device of claim 10, wherein the predefined time period is 20 minutes.

13. The detecting device of claim 10, wherein the predetermined acceleration threshold is 20 cm/s².

14. The detecting device of claim 9, wherein the variation ratio is 20%.

15. The detecting device of claim 9, wherein the predetermined speed is 10 km/h, and the predetermined ratio is 1/6.

16. The detecting device of claim 9, wherein the variation threshold of each of the exercise modes is 10%.
